(19) United States
(12) Patent Application Publication
(10) Pub. No.: US 2024/0153075 A1
(43) Pub. Date: May 9, 2024

(54) ELECTRONIC DEVICE FOR AI-BASED RECOMMENDATION OF MELANOMA BIOPSY SITE AND METHOD FOR PERFORMING THE SAME

(71) Applicant: THE CATHOLIC UNIVERSITY OF KOREA INDUSTRY-ACADEMIC COOPERATION FOUNDATION, Seoul (KR)

(72) Inventors: Ju Hee HAN, Seoul (KR); Ji Ho PARK, Seoul (KR); Young-Min PARK, Suwon (KR); Ji-Hyun LEE, Seoul (KR); Chul Hwan BANG, Seoul (KR); Woo Hyup LEE, Seoul (KR); Jeong Wook MOON, Seoul (KR)

(21) Appl. No.: 18/385,925

(22) Filed: Nov. 1, 2023

(30) Foreign Application Priority Data
Nov. 3, 2022 (KR) 10-2022-0145165

(51) Int. Cl. G06T 7/00; G06T 7/90

(52) U.S. Cl. CPC G06T 7/0012; G06T 7/90; G06T 2207/20081; G06T 2207/30088

(57) ABSTRACT

The electronic device for recommending an AI-based melanoma biopsy site according to an exemplary embodiment of the present invention includes a processor which classifies a skin image that is input by using a classification model as melanoma or nevus, identifies melanoma features in the skin image by using a generation model when the skin image is classified as melanoma to generate an image from which the melanoma features are removed, and compares the skin image with the generated image to identify at least one candidate biopsy site.

### CROSS-REFERENCE TO RELATED APPLICATION

This application claims priority to and the benefit of Korean Patent Application No. 10-2022-0145165, filed on Nov. 3, 2022, the disclosure of which is incorporated herein by reference in its entirety.

### TECHNICAL FIELD

The present invention relates to an electronic device and method for recommending a melanoma biopsy site.

### BACKGROUND ART

Melanoma is a malignant tumor arising from melanocytes, mainly on the skin. Among various types of skin cancer, melanoma is potentially the most dangerous type and accounts for approximately 90% of skin cancer deaths. The World Health Organization (WHO) predicts that the frequency and death toll of melanoma will increase steadily through 2040, but the good news is that melanoma has a good prognosis if it is diagnosed early.

Early diagnosis and primary access are important to achieve a positive prognosis in melanoma. Biopsy is the best way to diagnose skin cancer. However, since biopsy is an invasive test, complications may occur, and false negative results may occur depending on inappropriate biopsy.

As a method of biopsy, excisional biopsy is used as the standard diagnostic method, in which the entire tumor and normal tissue are removed and the tissue is harvested. If the lesion is large or skin relaxation is limited, incisional biopsy or punch biopsy is often used According to one study, the result was reported that the false negative rate of punch biopsy among melanoma biopsies conducted at tertiary medical institutions was 23.3%, which is very high compared to 1.7% and 4.5% of other excisional biopsies and shave biopsies, respectively. In spite of the high false negative rate of punch biopsy, it is still frequently used in most institutions due to its convenience.

Meanwhile, dermoscopy is a non-invasive diagnostic tool that can clarify the differential diagnosis of pigmented lesions and compensate for the disadvantages of punch biopsy. The appropriate usage of dermoscopy may lead to higher sensitivity and specificity in the diagnosis of melanoma. However, depending on the user's experience, the accuracy may be lowered, and there is a risk of misdiagnosis.

Therefore, it is very important to find and examine a suitable biopsy site in order to obtain an appropriate result by using an appropriate biopsy method depending on the situation as well as considering the versatility of punch biopsy.

### DISCLOSURE

### Technical Problem

An object of the present invention is to provide an electronic device and method for recommending a melanoma biopsy site to obtain more accurate biopsy results.

Another object of the present invention is to provide an electronic device and method for recommending a melanoma biopsy site as a non-invasive supplement to biopsy.

### Technical Solution

The electronic device for recommending an AI-based melanoma biopsy site according to an exemplary embodiment of the present invention includes a processor which classifies a skin image that is input by using a classification model as melanoma or nevus, identifies melanoma features in the skin image by using a generation model when the skin image is classified as melanoma to generate an image from which the melanoma features are removed, and identifies at least one candidate biopsy site by comparing the skin image with the generated image.

The processor may identify pixel values according to color of pixels of the skin image and the generated image, and identify at least one candidate biopsy site by comparing pixel values of corresponding pixels of the skin image and the generated image.

The processor may identify the pixel values as three-dimensional coordinate values having R (Red), G (Green) and B (Blue) as axes, respectively, according to the RGB values of the color of each pixel.

The processor may calculate a distance between pixels for each pixel by using coordinate values of corresponding pixels of the skin image and the generated image, and identify a predefined number of pixels as the at least one candidate biopsy site based on the distance between pixels.

The processor may map and display a priority for each candidate biopsy site on the at least one candidate biopsy site of the skin image.

The processor may use the skin image and the generated image by filtering with a Gaussian filter.

The classification model may be learned to classify whether an input skin image is melanoma or nevus by using, as learning data, a plurality of skin images including a plurality of melanoma images and a plurality of nevus images and answer information on whether each skin image is melanoma or nevus.

The generation model may be learned to identify melanoma features and nevus features from a plurality of skin images including a plurality of melanoma images and a plurality of nevus images and to generate an image in which the melanoma features are removed from the melanoma images.

The method for recommending an AI-based melanoma biopsy site which is performed by an electronic device according to an exemplary embodiment of the present invention includes the steps of classifying a skin image that is input by using a classification model as melanoma or nevus; identifying melanoma features in the skin image by using a generation model when the skin image is classified as melanoma to generate an image from which the melanoma features are removed; and identifying at least one candidate biopsy site by comparing the skin image with the generated image.

The step of identifying at least one candidate biopsy site may include the steps of identifying pixel values according to color of pixels of the skin image and the generated image; and identifying at least one candidate biopsy site by comparing pixel values of corresponding pixels of the skin image and the generated image.

The step of identifying pixel values may include the step of identifying the pixel values as three-dimensional coordinate values having R (Red), G (Green) and B (Blue) as axes, respectively, according to the RGB values of the color of each pixel. The step of identifying at least one candidate biopsy site may include the steps of calculating a distance between pixels for each pixel by using coordinate values of corresponding pixels of the skin image and the generated image; and identifying a predefined number of pixels as the at least one candidate biopsy site based on the distance between pixels.

The method may further include the step of mapping and displaying a priority for each candidate biopsy site on the at least one candidate biopsy site of the skin image.

The step of identifying at least one candidate biopsy site may include the step of using the skin image and the generated image by filtering with a Gaussian filter.

### Advantageous Effects

According to an exemplary embodiment of the present invention, it is possible to identify a suitable candidate biopsy site using only skin images by utilizing artificial intelligence models, and thus, it is possible to supplement a non-invasive method for early diagnosis of melanoma.

According to an exemplary embodiment of the present invention, the accuracy of early diagnosis of melanoma can be increased through more accurate identification of candidate biopsy locations.

### DESCRIPTION OF DRAWINGS

FIG. 1 is a schematic diagram illustrating the process of recommending a melanoma biopsy site according to an exemplary embodiment of the present invention.

FIG. 2 is a block diagram illustrating the configuration of an electronic device according to an exemplary embodiment of the present invention.

FIG. 3 is a diagram illustrating the operation flowchart of an electronic device according to an exemplary embodiment of the present invention.

FIG. 4 is a diagram illustrating the operation status of an electronic device according to an exemplary embodiment of the present invention.

FIG. 5 is a diagram illustrating the learning process of a generation model according to an exemplary embodiment of the present invention.

FIG. 6 is a diagram illustrating a skin image of melanoma and a generated image corresponding thereto according to an exemplary embodiment of the present invention.

FIG. 7 is a diagram illustrating a recommended biopsy site image according to an exemplary embodiment of the present invention.

### MODES OF THE INVENTION

Hereinafter, preferred exemplary embodiments according to the present invention will be described in detail with reference to the accompanying drawings.

The detailed description set forth below in conjunction with the accompanying drawings is intended to describe the exemplary embodiments of the present invention and is not intended to represent the only exemplary embodiments in which the present invention may be practiced. In order to clearly describe the present invention in the drawings, parts that are irrelevant to the description may be omitted, and the same reference numerals may be used for the same or similar components throughout the specification.

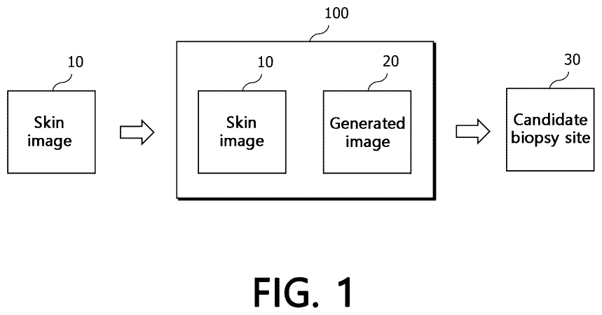

FIG. 1 is a schematic diagram illustrating the process of recommending a melanoma biopsy site according to an exemplary embodiment of the present invention.

Referring to FIG. 1, the electronic device 100 according to an exemplary embodiment of the present invention is a device that recommends a suitable biopsy site by using an input skin image 10, and it may be implemented as a computer, a server or the like.

The skin image 10 according to an exemplary embodiment of the present invention may be an image acquired by photographing an affected area for diagnosing skin cancer of a patient. However, the method and route for acquiring the skin image 10 do not limit the present invention.

In this case, the affected area on the skin image 10 may be melanoma or nevus. Hereinafter, the fact that whether the skin image 10 is melanoma or nevus is used to indicate whether the affected area on the skin image 10 is melanoma or nevus.

The operation of identifying whether the skin image 10 is melanoma or nevus may be performed by an artificial intelligence model, and the details thereof will be described below.

If it is a nevus, the electronic device 100 does not perform an additional operation, but if it is a melanoma, the electronic device 100 generates a generated image 20 by using the skin image 10 to recommend a suitable biopsy site.

Specifically, the electronic device 100 generates an image close to the skin image 10, but generates a generated image 20 from which melanoma features on the skin image 10 are removed. The process of generating a generated image 20 may also be performed by an artificial intelligence model, such as a model for identifying melanoma and nevus, and the details thereof will be described below.

In addition, the electronic device 100 compares the skin image 10 having melanoma features and the generated image 20 having no melanoma features, and recommends a location with a large difference between the images as a candidate biopsy site 30.

The candidate biopsy site 30 means a site that is recommended for biopsy in the affected area in the skin image 10. The candidate biopsy site 30 is determined according to criteria for comparing the skin image 10 and the generated image 20, and it may be one or more.

As such, the present invention proposes an electronic device and method for recommending a biopsy site based on a skin image in order to overcome the above-mentioned limitations of early diagnosis of melanoma and help a primary medical approach. Through this, it is possible to apply non-invasive and augmented approaches to conventional biopsies.

Moreover, by implementing a technique that recommends a melanoma biopsy site based on artificial intelligence technology, the versatility of usage may be expected, such as deriving results that are equal to or higher than the level of decision-making by medical staff, improving inefficiency of the medical delivery system, or utilizing in areas with insufficient medical resources.

Hereinafter, the configuration and operation of an electronic device according to an exemplary embodiment of the present invention will be described in detail with reference to the drawings.

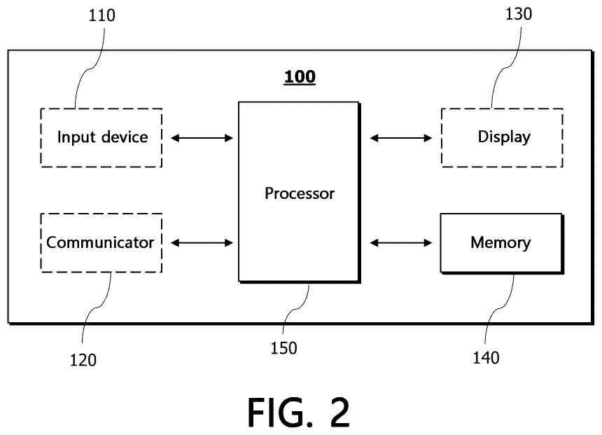

FIG. 2 is a block diagram illustrating the configuration of an electronic device according to an exemplary embodiment of the present invention.

The electronic device 100 according to an exemplary embodiment of the present invention includes an input device 110, a communicator 120, a display 130, a memory 140 and a processor 150.

The input device 110 generates input data in response to a user input of the electronic device 100. For example, the user input may be a user input for starting an operation of the electronic device 100, a user input for loading a skin image and the like, and in addition to the above, when it is a user input that is required to identify a candidate biopsy site by using a skin image, it may be applied without limitation.

The input device 110 includes at least one input means. The input device 110 may include a keyboard, a key pad, a dome switch, a touch panel, a touch key, a mouse, a menu button and the like.

The communicator 120 communicates with an external device such as a photographing device such as a skin magnifying glass and a server in order to receive a skin image, a classification model, a generation model and the like. To this end, the communicator 120 may perform wireless communication such as 5th generation communication (5G), long term evolution-advanced (LTE-A), long term evolution (LTE), wireless fidelity (Wi-Fi) and the like, or wired communication such as a local area network (LAN), wide area network (WAN), power line communication and the like.

The display 130 displays display data according to the operation of the electronic device 100. The display 130 may display a screen for displaying a skin image and a generated image, a screen for displaying a candidate biopsy site, a screen for receiving a user input and the like.

The display 130 may include a liquid crystal display (LCD), a light emitting diode (LED) display, an organic LED (OLED) display, a micro-electro mechanical systems (MEMS) display and an electronic paper display. The display 130 may be combined with the input device 110 and implemented as a touch screen.

The memory 140 stores operating programs of the electronic device 100. The memory 140 includes a non-volatile storage that can retain data (information) regardless of whether electric power is provided, and a volatile memory that can load the data to be processed by the processor 150 and cannot retain data if electric power is not provided. Examples of the storage include flash-memory, hard-disc drive (HDD), solid-state drive (SSD), read-only memory (ROM) and the like, and examples of the memory include a buffer, a random access memory (RAM) and the like.

The memory 140 may store operation programs and the like that are required in the processes of storing a skin image, a generated image, information on a candidate biopsy site, a classification model, a generated model and the like that are received from an external device, classifying whether the skin image is melanoma or nevus, identifying melanoma features to generate an image from which melanoma features are removed, identifying at least one candidate biopsy site by comparing the skin image with the generated image, and performing the learning of a classification model and a generation model.

The processor 150 may execute software such as a program to control at least one other component (e.g., a hardware or software component) of the electronic device 100, and perform various data processing or calculations. According to an exemplary embodiment, the processor 150 may include a main processor for serving as a central processing unit (CPU) or an application processor, and a graphic processing unit (GPU) that can be operated independently or together with the main processor.

The processor 150 classifies whether the input skin image is melanoma or nevus by using a classification model, identifies melanoma features in the skin image by using a generation model when the skin image is classified as melanoma to generate an image from which the melanoma features are removed, and identifies at least one candidate biopsy site by comparing the skin image with the generated image.

In this case, the processor 150 may learn a classification model and/or a generation model, or receive and store a previously learned classification model and/or generation model from the outside and use the same, but the present invention is not limited thereto.

Meanwhile, the processor 150 may perform at least some of the data analysis, processing and result information generation for performing the above operations by using at least one of machine learning, neural network or deep learning as a rule-based or artificial intelligence algorithm. Examples of the neural network may include models such as Deep Convolutional Neural Network (DCNN), Convolutional Neural Network (CNN), Deep Neural Network (DNN), Recurrent Neural Network (RNN) and Generative Adversarial Networks (GAN).

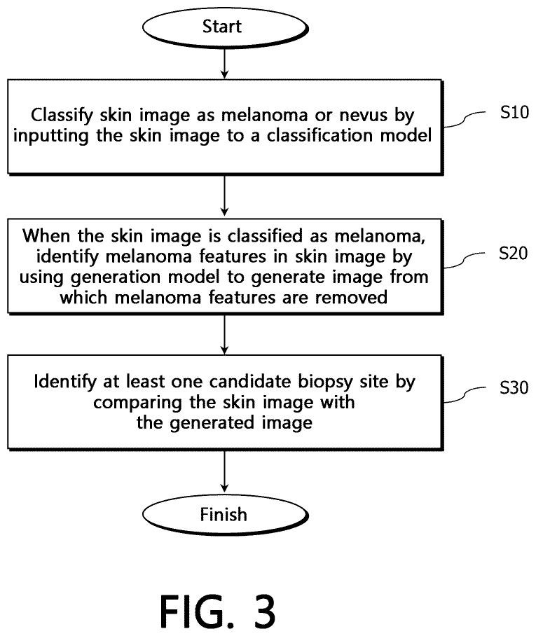

FIG. 3 is a diagram illustrating the operation flowchart of an electronic device according to an exemplary embodiment of the present invention.

According to an exemplary embodiment of the present invention, the processor 150 classifies whether the input skin image is melanoma or nevus by using a classification model (S10).

The classification model according to an exemplary embodiment of the present invention is a model which is learned to classify whether a skin image is a melanoma or a nevus. Specifically, the classification model is characterized in that it is learned to classify an input skin image as melanoma or nevus by using, as learning data, a plurality of skin images and answer information on whether each skin image is melanoma or nevus. The classification model may be learned by using Deep Convolutional Neural Network (DCNN).

In this case, as described above, the processor 150 may learn a classification model or may receive and store a classification model generated through pre-learning from the outside and use the same. A case where the processor 150 learns a classification model will be described.

The plurality of skin images may include a plurality of melanoma images and a plurality of nevus images, and the plurality of skin images may include a public dataset (HAM10000 melanoma dataset) or data accumulated through treatment at a hospital. The processor 150 may exclude inappropriate skin images, for example, scalp lesions or low-quality images, among the collected data from the learning data. The processor 150 may adjust the plurality of skin images to a size that is suitable for the classification model, for example, 224×224 pixels (but the present invention is not limited thereto).

The processor 150 may generate learning data by labeling each skin image with correct answer information. In this case, the correct answer information is information indicating whether each skin image is melanoma or nevus.

The processor 150 may train a classification model to classify whether an input skin image is melanoma or nevus by using the learning data.

According to an exemplary embodiment of the present invention, when the skin image is classified as melanoma, the processor 150 identifies melanoma features in the skin image by using a generation model to generate an image (generated image) from which the melanoma features are removed (S20).

Meanwhile, when the skin image is classified as a nevus, the processor 150 may display the classification result as a final result on the display 130 or transmit the result to a server or an external device that requests the result through the communicator 120.

When the skin image is classified as melanoma through the classification model, the processor 150 inputs the corresponding skin image to a generation model.

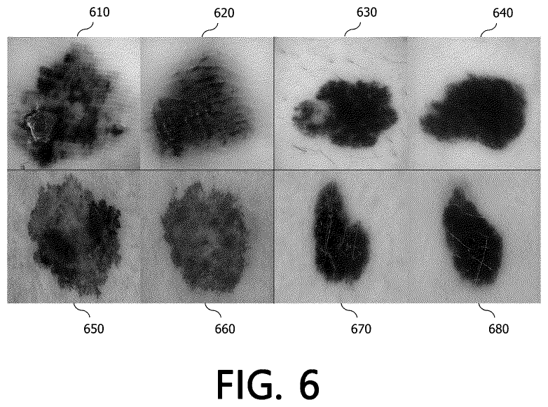

The generation model according to an exemplary embodiment of the present invention is a model which is learned to generate an image similar to an input skin image, but with melanoma features removed from the skin image. The generation model may be trained to mimic the morphological characteristics of a nevus. That is, when the input skin image is a melanoma image, the generation model generates a nevus image that imitates the input skin image. Examples of skin images and generated images corresponding thereto are shown in FIG. 6.

Specifically, the generation model is characterized in that it is learned to identify melanoma features and nevus features from a plurality of skin images including a plurality of melanoma images and a plurality of nevus images to generate an image in which the melanoma features are removed from the melanoma images.

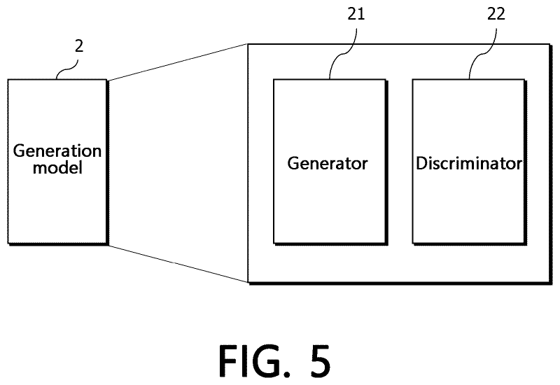

The generation model may be learned by using GAN, and especially, styleGAN2, and the specific learning process will be described with reference to FIG. 5.

According to an exemplary embodiment of the present invention, the processor 150 identifies at least one candidate biopsy site by comparing the skin image with the generated image (S30).

As described above, the processor 150 may compare a skin image having melanoma features with a generated image not having melanoma features, and recommend a location having a large difference between the images as a candidate biopsy site.

In this case, various methods may be tried to compare the difference between images, and the method of using pixel values for each pixel will be described as an example.

According to an exemplary embodiment of the present invention, the processor 150 may identify pixel values according to colors of pixels of the skin image and the generated image, and identify at least one candidate biopsy site by comparing pixel values of corresponding pixels of the skin image and the generated image.

Images such as skin images and generated images are composed of a plurality of pixels and may have pixel values according to the color of each pixel.

In this case, as one of the methods of expressing color, the processor 150 may identify the pixel values as three-dimensional coordinate values having R (Red), G (Green) and B (Blue) as axes, respectively, according to the RGB values of the color of each pixel. For example, if any one pixel of the skin image is white, the pixel value is (255, 255, 255).

In this way, the processor 150 may identify pixel values for each pixel of the images and compare pixel values of corresponding pixels of the skin image and the generated image.

The corresponding pixels of the skin image and the generated image refer to pixels located at the same location in each image, when the sizes of the skin image and the generated image are the same. If the sizes are different, the processor 150 may identify pixels at corresponding locations on each image or may identify pixels at the same location by adjusting the sizes of the images to be the same.

As one of the methods of comparing pixel values, the processor 150 may use a method of calculating a distance between pixels for each pixel by using the coordinate values of corresponding pixels of the skin image and the generated image. As described above, the processor 150 may calculate a distance between pixels by using two coordinate values, because each pixel is represented by an RGB coordinate value.

The process 150 may identify, as at least one candidate biopsy site, pixels corresponding to the longest distances among the calculated distances between pixels. In this case, a predefined number of candidate biopsy sites may be identified, and the processor 150 may identify candidate biopsy sites through a user input for designating a predefined number received through the input device 110.

As an additional example, the processor 150 may use the skin image and the generated image by filtering with a Gaussian filter. The Gaussian filter is a filter mask that is created by approximating the Gaussian distribution function, and although the difference in pixel values does not occur according to the melanoma features, it is used to prevent having a non-ideal value from any one specific pixel for various reasons such as noise and the like. Accordingly, the processor 150 may calculate a distance between pixels after filtering each image with a Gaussian filter.

Afterwards, the processor 150 may display the identified candidate biopsy site.

There may be various methods of displaying the same, and for example, the processor 150 may map and display a priority for each candidate biopsy site on at least one candidate biopsy site of the skin image. For example, if five candidate biopsy sites are identified, each site may be marked with a red dot and a number between 1 and 5 next to each dot.

As another example, the processor 150 may generate a distance map in which values according to the distance between pixels for each pixel are mapped, and display the skin image in color or contrast according to the distance between pixels. For example, when it is displayed in a darker color if the distance is far and is displayed in a brighter color if the distance is close, the candidate biopsy sites may be identified as regions.

According to an exemplary embodiment of the present invention, a suitable candidate biopsy site may be identified by using only skin images utilizing artificial intelligence models, and thus, it is possible to supplement the early diagnosis of melanoma with a non-invasive method.

According to an exemplary embodiment of the present invention, the accuracy of early diagnosis of melanoma may be increased through more accurate identification of candidate biopsy locations.

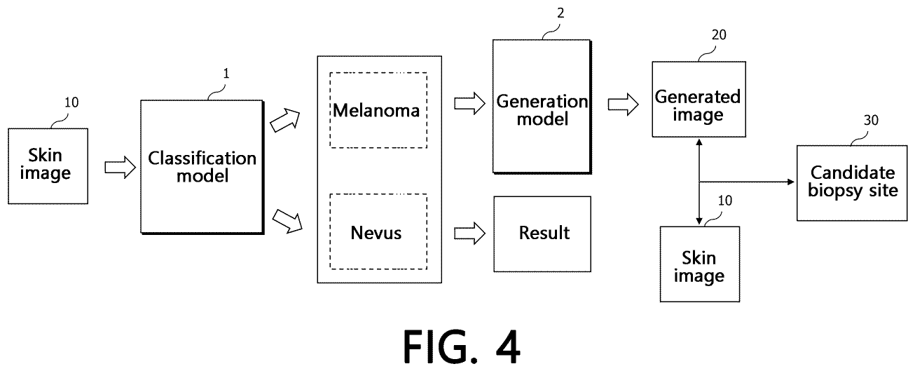

FIG. 4 is a diagram illustrating the operation status of an electronic device according to an exemplary embodiment of the present invention.

In FIG. 4, as described with reference to FIG. 3, the process of identifying candidate biopsy locations is generally described. In this case, the detailed description of the contents overlapping with the contents described in FIG. 3 will be omitted.

First of all, when the skin image 10 is input to a classification model 1, the classification model 1 classifies the skin image 10 as melanoma or nevus.

When the skin image 10 is a nevus, information indicating that the corresponding image is a nevus (benign tumor) may be output.

When the skin image 10 is a melanoma, the corresponding skin image 10 is input to the generation model 2. The skin image 10 is converted into the generated image 20 through the generation model 2, and the candidate biopsy site 30 may be identified by comparing the skin image 10 and the generated image 20.

FIG. 5 is a diagram illustrating the learning process of a generation model according to an exemplary embodiment of the present invention.

As described above in relation to S20 of FIG. 3, the generation model may be learned by using GAN, and especially, styleGAN2, and similarly, the processor 150 may learn the generation model or use by receiving and storing the previously learned and generated generation model from the outside.

GAN is an adversarial generative neural network, and it is one of artificial intelligence models that are designed for the purpose of generating an image similar to the original image by adversarial learning of two different networks, such as a generator 21 and a discriminator 22.

The generator 21 is learned to create an image similar to the original image, and the discriminator 22 is learned to distinguish whether the image generated by the generator 21 is real or fake. In this case, the GAN learns to create an image that is very similar to the original image while finding a balance point between the generator 21 and the discriminator 22. If the generator 21 creates an image that is very similar to the original image, the discriminator 22 will find it difficult to distinguish the same, and the generation model 2 repeats learning until the discriminator 22 cannot discriminate between the original image and the generated image.

In the present invention, based on the skin image that is determined to be melanoma, the generation model which is learned to generate an image that mimics the characteristics of a nevus while looking similar is used.

As the learning data, as described above in S10 of FIG. 3, a plurality of skin images used in the classification model or a plurality of skin images acquired in the same way as the classification model may be used. In addition, the processor 150 may adjust the plurality of skin images to a size suitable for the generation model, for example, 1,024×1,024 pixels (but the present invention is not limited thereto).

Morphological differences between nevus and melanoma show differences in shape, pigmentation pattern, pigmentation pattern in ridges and furrows, color and the like.

The processor 150 may identify melanoma features and nevus features based on these differences, learn the identified melanoma features and nevus features, and train a generation model to generate an image in which the melanoma features are removed from the skin image.

Therefore, when a skin image of melanoma is input to the generation model, an image without melanoma features is generated as if it were a nevus image closest to the input image.

According to one example, it may be designed to include an algorithm for recommending candidate biopsy sites in the latter part of the network of generation models.

FIG. 6 is a diagram illustrating a skin image of melanoma and a generated image corresponding thereto according to an exemplary embodiment of the present invention.

Referring to FIG. 6, melanoma skin images 610, 630, 650, 670 and generated images 620, 640, 660, 680 corresponding to the respective skin images are paired and arranged.

When the skin image 610 and the generated image 620 corresponding thereto are compared, it can be confirmed that the blue-whitish pattern of the skin image 610 and the part accompanied by the ulcer disappear from the generated image 620, and the pigmentation part that is observed as a ridge pattern of the skin image 610 appears to be centered on a furrow pattern in the generated image 620 When the skin image 630 and the generated image 640 corresponding thereto are compared, it can be seen that the overall shape of the lesion in the skin image 630 is irregular, and a part on the left side is observed to be pink, indicating that it is accompanied by an ulcer, whereas in the generated image 640, this part disappears, and regular pigmentation in a uniform form appears.

When the skin image 650 and the generated image 660 corresponding thereto are compared, it can be seen that the overall shape of the skin image 650 is irregular, and the pigmentation pattern is quite non-uniform, and in the generated image 660, it can be seen that the overall shape and pigmentation pattern are uniformly formed.

Finally, when the skin image 670 and the generated image 680 corresponding thereto are compared, in the skin image 670, it can be confirmed that the lesions showing relatively irregular shapes and pigments are expressed more uniformly in the generated image 680.

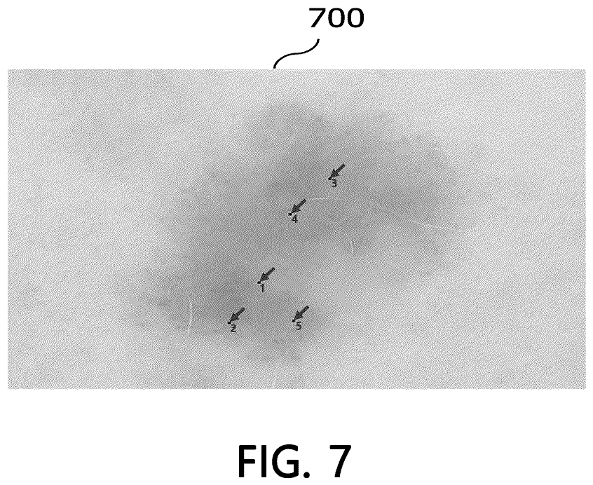

FIG. 7 is a diagram illustrating a recommended biopsy site image according to an exemplary embodiment of the present invention.

FIG. 7 illustrates an example in which the processor 150 displays the identified candidate biopsy site on the skin image, as described in relation to S30 of FIG. 3.

In the skin image 700 shown in FIG. 7, five identified candidate biopsy sites are marked with red dots, and a number between 1 and 5 is assigned next to each dot.

However, the skin image 700 in which the candidate biopsy sites are displayed in FIG. 7 is only an example, and the method of displaying the candidate biopsy sites on the skin image may be applied without limitation.

Based on the skin image 700, the medical personnel may perform a biopsy from a site with the highest priority, or may perform a biopsy from a site that is determined to be most suitable among candidate biopsy sites.

### EXPLANATION OF REFERENCE NUMERALS

100: Electronic device
110: Input device
120: Communicator
130: Display
140: Memory
150: Processor

1. An electronic device for AI-based recommendation of a melanoma biopsy site, comprising:
a processor configured to:
classify a skin image as melanoma or nevus by inputting the skin image to a classification model,
when the skin image is classified as melanoma, identify melanoma features in the skin image by using a generation model to generate an image from which the melanoma features are removed, and
identify at least one candidate biopsy site by comparing the skin image with the generated image.

2. The electronic device of claim 1, wherein the processor is configured to identify pixel values according to color of pixels of the skin image and the generated image, and identify the at least one candidate biopsy site by comparing the pixel values of corresponding pixels of the skin image and the generated image.

3. The electronic device of claim 2, wherein the processor is configured to identify the pixel values as three-dimensional coordinate values having R (Red), G (Green) and B (Blue) as axes, respectively, according to the RGB values of the color of each pixel.

4. The electronic device of claim 3, wherein the processor is configured to calculate a distance between pixels for each pixel by using coordinate values of corresponding pixels of the skin image and the generated image, and identify a predefined number of pixels as the at least one candidate biopsy site based on the distance between pixels.

5. The electronic device of claim 4, wherein the processor is configured to map and display a priority for each candidate biopsy site on the at least one candidate biopsy site of the skin image.

6. The electronic device of claim 4, wherein the processor is configured to use the skin image and the generated image by filtering with a Gaussian filter.

7. The electronic device of claim 1, wherein the classification model is learned to classify whether a skin image being input is melanoma or nevus by using, as learning data, a plurality of skin images including a plurality of melanoma images and a plurality of nevus images and answer information on whether each skin image is melanoma or nevus.

8. The electronic device of claim 1, wherein the generation model is learned to identify melanoma features and nevus features from a plurality of skin images including a plurality of melanoma images and a plurality of nevus images and to generate an image in which the melanoma features are removed from the melanoma images.

9. A method for AI-based recommendation of a melanoma biopsy site which is performed by an electronic device, comprising the steps of:
classifying a skin image as melanoma or nevus by inputting the skin image to a classification model;
when the skin image is classified as melanoma, identifying melanoma features in the skin image by using a generation model to generate an image from which the melanoma features are removed; and
identifying at least one candidate biopsy site by comparing the skin image with the generated image.

10. The method of claim 9, wherein the step of identifying at least one candidate biopsy site comprises the steps of:
identifying pixel values according to color of pixels of the skin image and the generated image; and
identifying the at least one candidate biopsy site by comparing the pixel values of corresponding pixels of the skin image and the generated image.

11. The method of claim 10, wherein the step of identifying pixel values comprises the step of:
identifying the pixel values as three-dimensional coordinate values having R (Red), G (Green) and B (Blue) as axes, respectively, according to the RGB values of the color of each pixel.

12. The method of claim 11, wherein the step of identifying at least one candidate biopsy site comprises the steps of:
calculating a distance between pixels for each pixel by using coordinate values of corresponding pixels of the skin image and the generated image; and
identifying a predefined number of pixels as the at least one candidate biopsy site based on the distance between pixels.

13. The method of claim 12, further comprising the step of:
mapping and displaying a priority for each candidate biopsy site on the at least one candidate biopsy site of the skin image.

14. The method of claim 12, wherein the step of identifying at least one candidate biopsy site comprises the step of:
using the skin image and the generated image by filtering with a Gaussian filter.

15. The method of claim 9, wherein the classification model is learned to classify whether a skin image being input is melanoma or nevus by using, as learning data, a plurality of skin images including a plurality of melanoma images and a plurality of nevus images and answer information on whether each skin image is melanoma or nevus.

16. The method of claim 9, wherein the generation model is learned to identify melanoma features and nevus features from a plurality of skin images including a plurality of melanoma images and a plurality of nevus images and to generate an image in which the melanoma features are removed from the melanoma images.
